(19) United States
(12) Patent Application Publication
(10) Pub. No.: US 2021/0379012 A1
(43) Pub. Date: Dec. 9, 2021

(54) ANTIANGIOGENIC CHROMANE DERIVATIVE AND USES THEREOF

(71) Applicants: Indiana University Research and Technology Corporation, Indianapolis, IN (US); Gachon University of Industry-Academic Cooperation Foundation, Seongnam-si, Gyeonggi-do (KR); KINGSTON UNIVERSITY, Kingston upon Thames, Surrey (GB); UNIVERSITY OF SURREY, Guildford, Surrey (GB)

(72) Inventors: Timothy W. Corson, Fishers, IN (US); Seung-Yong SEO, Seongnam-si (KR); Bit Lee, Saongnam-si (KR); Sianne SCHWIKKARO, Surrey (GB); Dulcie MULHOLLAND, Surrey (GB); Hannah WHITMORE, Surrey (GB)

(21) Appl. No.: 17/051,807

(22) PCT Filed: Apr. 29, 2019
(86) PCT No.: PCT/KR2019/005143

§ 371 Date: Oct. 30, 2020

Related U.S. Application Data
(60) Provisional application No. 62/664,584, filed on Apr. 30, 2018.

(51) Int. Cl. A61K 31/353; C07D 309/06; A61K 31/7072; A61K 38/17; A61K 39/395; A61K 31/506; A61K 31/44; A61K 31/427

(52) U.S. Cl. CPC A61K 31/353; C07D 309/06; A61K 31/7072; A61K 31/427; A61K 39/3955; A61K 31/506; A61K 31/44; A61K 38/179

(57) ABSTRACT

A novel chromane derivative, (3′-hydroxy-4′-ethoxybenzyl)-5,6,7-trimethoxy-2H-1-benzopyran (SH-17059 (1)) is disclosed herein. This chromane derivative has been found to inhibit blood vessel cell growth and proliferation, providing a promising treatment for ocular angiogenesis-mediated diseases.

### STATEMENT OF GOVERNMENT SUPPORT

This invention was made with government support under EY025641 awarded by National Institutes of Health. The government has certain rights in the invention.

### TECHNICAL FIELD

The present disclosure relates generally to a new chromane derivative developed and found to be a potent inhibitor of the growth of human retinal endothelial cells. Accordingly, the new derivative compound has utility as a new treatment for angiogenesis-mediated diseases, particularly, angiogenesis-medicated diseases such as retinopathy of prematurity (ROP), proliferative diabetic retinopathy (PDR) and the wet form of age-related macular degeneration (AMD).

### BACKGROUND ART

Abnormal formation of new blood vessels in the eye is associated with blindness in many ocular diseases such as retinopathy of prematurity (ROP), proliferative diabetic retinopathy (PDR) and the wet form of age-related macular degeneration (AMD), affecting children, adults, and elderly people, respectively. The newly formed vasculature is fragile and leaky, causing hemorrhage and accumulation of fluids in the retina. If left untreated, the resulting fibrotic scarring can lead to irreversible vision loss.

The current pharmacotherapeutic mainstays for these diseases are biologics targeting the vascular endothelial growth factor (VEGF) such as bevacizumab, ranibizumab, and pegaptanib. Despite being successful in suppressing disease progression, these large molecule therapies are associated with some undesirable ocular and systemic side effects. Moreover, resistance is a problem: about 30% of wet AMD patients are resistant to these biologics. Currently, there is no FDA approved small molecule for the treatment of ocular neovascularization.

### DISCLOSURE OF INVENTION

### Technical Problem

Based on the foregoing, there is an unmet need to develop novel and specific antiangiogenic small molecule therapies to complement and combine with existing drugs for ROP, PDR, and wet AMD.

### Solution to Problem

The present disclosure is generally directed to a new chromane derivative found to have potential as a new treatment for angiogenesis-mediated diseases. Particularly, the chromane derivative (1) has been found to be a potent inhibitor of the growth of human retinal endothelial cells, and thus, has potential to treat ocular angiogenesis-mediated diseases.

Accordingly, in one aspect, the present disclosure is directed to a chromane derivative of formula (1)

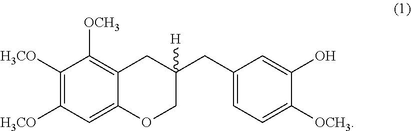 [SMILES: COc1ccc(CC2COc3cc(OC)c(OC)c(OC)c3C2)cc1O]

In another aspect, the present disclosure is directed to a method of inhibiting cell growth, the method comprising contacting the cell with a chromane derivative of formula (1).

In yet another aspect, the present disclosure is directed to a method of treating or preventing ocular angiogenesis-mediated disease in a subject in need thereof, the method comprising administering to the subject a chromane derivative of formula (1). Particularly diseases for treatment or prevention include, for example, angiogenesis-medicated diseases such as retinopathy of prematurity (ROP), proliferative diabetic retinopathy (PDR), the wet form of age-related macular degeneration (AMD), pathological myopia, hypertensive retinopathy, occlusive vasculitis, polypoidal choroidal vasculopathy, diabetic macular edema, uveitic macular edema, central retinal vein occlusion, branch retinal vein occlusion, corneal neovascularization, retinal neovascularization, ocular histoplasmosis, neovascular glaucoma, retinoblastoma, and combinations thereof.

In another aspect, the present disclosure is directed to a pharmaceutical composition comprising the chromane derivative of formula (1) for treatment or prevention of ocular angiogenesis-mediated disease. Particularly diseases for treatment or prevention include, for example, angiogenesis-medicated diseases such as retinopathy of prematurity (ROP), proliferative diabetic retinopathy (PDR), the wet form of age-related macular degeneration (AMD), pathological myopia, hypertensive retinopathy, occlusive vasculitis, polypoidal choroidal vasculopathy, diabetic macular edema, uveitic macular edema, central retinal vein occlusion, branch retinal vein occlusion, corneal neovascularization, retinal neovascularization, ocular histoplasmosis, neovascular glaucoma, retinoblastoma, and combinations thereof.

### BRIEF DESCRIPTION OF DRAWINGS

The disclosure will be better understood, and features, aspects and advantages other than those set forth above will become apparent when consideration is given to the following detailed description thereof. Such detailed description makes reference to the following drawings, wherein:

FIG. 1 shows that SH-17059 (1) potently blocks proliferation of human retinal endothelial cells (HRECs), alamarBlue proliferation assay results shown.

### BEST MODE FOR CARRYING OUT THE INVENTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

The present disclosure has identified a novel chromane derivative, (3′-hydroxy-4′-ethoxybenzyl)-5,6,7-trimethoxy-2H-1-benzopyran (1) (also known as “HW-1B” or “SH-17059” and referred to herein as“HW-1B” or “SH-17059”), having the formula of formula (1). This chromane derivative has been found to inhibit blood vessel cell growth and proliferation, providing a promising treatment for angiogenesis-mediated diseases. SH-17059 selectively inhibited the growth of endothelial cells, and in particular, human endothelial cells. The method for synthesizing SH-17059 is disclosed the Example below.

In some embodiments, SH-17059 is contacted with a cell to inhibit cell growth and proliferation. In particular embodiments, the cell is contacted with from about 0.01 nM to about 1 μM.

The chromane derivative compound, 2(3′-hydroxy-4′-ethoxybenzyl)-5,6,7-trimethoxy-2H-1-benzopyran (1) (SH-17059), can be used in methods for inhibiting cell growth, and particularly, blood vessel cell growth, and thereby treating ocular angiogenesis-mediated diseases. SH-17059 can be administered as a pharmaceutical composition comprising the derivative compound itself or in combination with one or more pharmaceutically acceptable carriers. As used herein, the phrase “pharmaceutically acceptable” refers to those ligands, materials, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier”, as used herein, refers to a pharmaceutically acceptable material, formulation or vehicle, such as a liquid or solid filler, diluent, excipient, solvent or encapsulating material, involved in carrying or transporting the active compound from one organ or portion of the body, to another organ or portion of the body. Each carrier must be “acceptable” in the sense of being compatible with the other components of the composition (e.g., synthetic compound) and not injurious to the subject. Lyophilized compositions, which may be reconstituted and administered, are also within the scope of the present disclosure.

Pharmaceutically acceptable carriers may be, for example, excipients, vehicles, diluents, and combinations thereof. For example, where the compositions are to be administered orally, they may be formulated as tablets, capsules, granules, powders, or syrups; or for parenteral administration, they may be formulated as injections (intramuscular, subcutaneous, intramedullary, intrathecal, intraventricular, intravenous, intravitreal), drop infusion preparations, or suppositories. For application by the ophthalmic mucous membrane route, they may be formulated as eye drops or eye ointments. These compositions can be prepared by conventional means, and, if desired, the active compound (i.e., SH-17059) may be mixed with any conventional additive, such as an excipient, a binder, a disintegrating agent, a lubricant, a corrigent, a solubilizing agent, a suspension aid, an emulsifying agent, a coating agent, or combinations thereof.

Suitable dosages of the chromane derivative compound (SH-17059) for use in the methods of the present disclosure will depend upon a number of factors including, for example, age and weight of an individual, severity of ocular disease, nature of a composition, route of administration and combinations thereof. Ultimately, a suitable dosage can be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

Administration of an effective amount of SH-17059 may be by a single dose, multiple doses, as part of a dosage regimen, and combinations thereof as determined by those skilled in the art for the relevant mechanism or process. The dosage regimen may vary depending on the symptoms, age and body weight of the subject, the nature and severity of the disorder to be treated or prevented, the route of administration and the form of the drug. In one particularly suitable embodiment, SH-17059 is administered as an eyedrop, twice daily. In one particularly suitable embodiment, SH-17059 is administered in a dosage ranging from about 0.2 μg to about 200 μg per eye.

It should be understood that the pharmaceutical compositions of the present disclosure can further include additional known therapeutic agents, drugs, modifications of the synthetic compounds into prodrugs, and the like for alleviating, mediating, preventing, and treating the diseases, disorders, and conditions described herein. For example, in one embodiment, SH-17059 can be administered with one or more anti-vascular endothelial growth factor (anti-VEGF) agents, including, but not limited to, pegaptanib, ranibizumab, aflibercept, bevacizumab, brolucizumab (also known as ESBA1008 and RTH258), conbercept (also known as KH-902), Abicipar Pegol, pazopanib, regorafenib, and PAN-90806 and combinations thereof.

The pharmaceutical compositions including SH-17059 and, optionally, additional therapeutic agents and pharmaceutical carriers, used in the methods of the present disclosure can be administered to a subset of subjects in need of treatment for ocular angiogenesis-mediated disease, including retinopathy of prematurity (ROP), proliferative diabetic retinopathy (PDR), diabetic retinopathy, wet age-related macular degeneration (AMD), pathological myopia, hypertensive retinopathy, occlusive vasculitis, polypoidal choroidal vasculopathy, diabetic macular edema, uveitic macular edema, central retinal vein occlusion, branch retinal vein occlusion, corneal neovascularization, retinal neovascularization, ocular histoplasmosis, neovascular glaucoma, retinoblastoma, and the like. Some subjects that are in specific need of treatment for ocular angiogenesis-mediated disease may include subjects who are susceptible to, or at elevated risk of, experiencing ocular disease (e.g., retinopathy of prematurity, diabetic retinopathy, “wet” age-related macular degeneration, etc.), and the like. Subjects may be susceptible to, or at elevated risk of, experiencing ocular diseases due to family history, age, environment, and/or lifestyle. Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions.

### MODE FOR THE INVENTION

Various functions and advantages of these and other embodiments of the present disclosure will be more fully understood from the examples shown below. The examples are intended to illustrate the benefits of the present disclosure, but do not exemplify the full scope of the disclosure.

### EXAMPLE

In this Example, (3′-hydroxy-4′-ethoxybenzyl)-5,6,7-trimethoxy-2H-1-benzopyran (1) (SH-17059) was synthesized and analyzed for its ability to inhibit cell growth and proliferation.

Synthesis of (1)

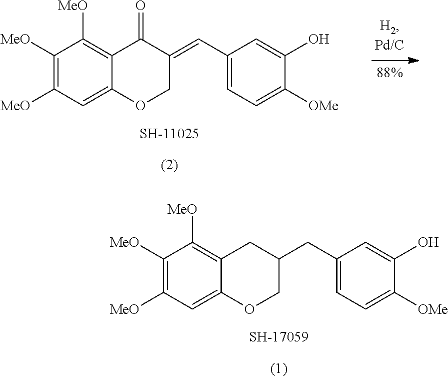 [SMILES: COc1ccc(/C=C2\COc3cc(OC)c(OC)c(OC)c3C2=O)cc1O ; COc1ccc(CC2COc3cc(OC)c(OC)c(OC)c3C2)cc1O]

(3′-hydroxy-4′-ethoxybenzyl)-5,6,7-trimethoxy-2H-1-benzopyran (1). (E)-3-(3-hydroxy-4-methoxybenzylidene)-5,6,7-trimethoxychroman-4-one (2) (SH-11025) has been synthesized before (Basavarajappa, H. D., et al., J Med Chem 58, 5015-5027, (2015)), and it was used as a starting point for synthesis of the novel chromane, (3′-hydroxy-4′-ethoxybenzyl)-5,6,7-trimethoxy-2H-1-benzopyran (1). Compound (2) was hydrogenated using Pd/C to give compound (1). Particularly, the anhydrous methanol solution of (E)-3-(3-hydroxy-4-methoxybenzylidene)-5,6,7-trimethoxychroman-4-one (2) (SH-11025) (22 mg, 0.059 mmol) and 10% Pd/C (6 mg) was placed under an atmosphere of hydrogen. After stirring for 24 hours, the reaction mixture was diluted with ethyl acetate, filtered through a Celite pad and concentrated under reduced pressure. The residue was purified by flash column chromatography on silica gel (ethyl acetate:n-hexane=1:2) to afford the hydrogenated compound (1) (19 mg, 88%). Yellow gum; UV (MeOH) λmax (log ε) 282 (−3.14) ¹H-NMR (600 MHz, CDCl₃) δ 6.79 (m, 1H), 6.69 (dd, 1H, J=7.8 and 1.8 Hz), 6.19 (s, 1H), 5.62 (s, 1H), 4.09 (m, 1H), 3.87 (s, 3H), 3.85 (s, 3H), 3.79 (s, 6H), 3.73 (dd, 1H, J=10.8 and 8.4 Hz), 2.77 (m, 1H), 2.59 (t, 2H, J=7.8 Hz), 2.34 (m, 1H), 2.22 (m, 1H); ¹³C-NMR (125 MHz, CDCl₃) δ 152.2, 151.6, 150.7, 145.5, 145.0, 135.8, 132.7, 120.4, 115.1, 110.6, 107.3, 95.9, 69.6, 61.0, 60.5, 56.0, 55.8, 37.6, 33.6, 25.5.

The synthesis reaction resulted in the reduction of both the 3,9-double bond as well as the carbonyl group at C-4 as shown by the loss of the fully-substituted carbon resonance at δ_{C} 179.8 (C-4) and the new methylene resonance at δ_{C} 37.5 (C-4). Coupling could clearly be seen in the COSY spectrum between the resonance at δH 2.14 (1H, m, H-3) and the resonance at δH 2.53 (2H, dd, J=4.1 and 7.4 Hz, H-4).

Proliferation Assays

EBM-2 and IMDM growth media were purchased from Lonza (Walkersville, Md., USA). RPMI and DMEM media were purchased from Thermo Scientific (Waltham, Mass., USA). ARPE19 cells were obtained from ATCC (Manassas, Va., USA). HRECs and Attachment Factor were purchased from Cell Systems (Kirkland, Wash., USA) and were used between passages 5 and 8. Endothelial Growth Medium (EGM-2) was prepared by mixing the contents of an EGM-2 “Bullet Kit” (Cat. no. CC-4176) with Endothelial Basal Medium (EBM) (Lonza). The EGM-2 “Bullet Kit” contains hydrocortisone, human fibroblast growth factor (hFGF), VEGF, R3-insulin like growth factor (R3-IGF-1), ascorbic acid, human epidermal growth factor (hEGF), gentamycin and heparin along with 2% fetal bovine serum (FBS). ARPE19 cells were grown in DEM medium containing 10% FBS and 1% penicillin-streptomycin (pen-strep). 92-1 cells were grown in RPMI medium containing 10% FBS and 1% pen-strep. Y79 cells were grown in RB medium (IMDM+10% FBS+55 μM β-mercaptoethanol+10 μg/mL Insulin+1% pen-strep). Identity of 92-1 and Y79 cell lines was confirmed by short tandem repeat profiling.

The proliferation of cells was monitored by an alamarBlue based fluorescence assay. Three cell types were used: HRECs, 92-1, and Y79. Briefly, 2,500 cells in 100 μL growth medium were incubated in 96-well clear bottom black plates for 24 hours followed by 48 hours' incubation with different concentrations of the test compound (range: 5 aM to 500 μM). At the end of the incubation, 11.1 μL of alamarBlue reagent was added and 4 hours after, fluorescence readings were taken with excitation and emission wavelengths of 560 nm and 590 nm respectively. Data were analyzed and dose response curves generated using GraphPad Prism software (v. 6.0).

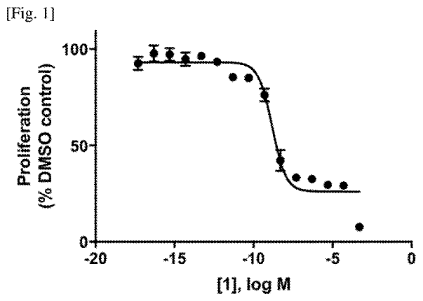

In a proliferation assay, compound (1) blocked the growth of HRECs with a GI₅₀ of 1.52 nM (FIG. 1). This far exceeds the potency of previously-disclosed antiangiogenic homoisoflavonoids. Moreover, compound (1) had very little effect on two other ocular cell lines, ARPE19 retinal pigment epithelial cells, 92-1 uveal melanoma cells and Y79 retinoblastoma cells, with GI₅₀=>100, 28, and 17 μM, respectively. This suggests remarkably good selectivity for endothelial cells rather than broad cytotoxicity.

1. A chromane derivative compound having the formula of formula (1):

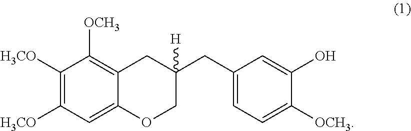 [SMILES: COc1ccc(CC2COc3cc(OC)c(OC)c(OC)c3C2)cc1O]

2. A method of inhibiting cell growth, the method comprising contacting the cell with a chromane derivative of formula (1):

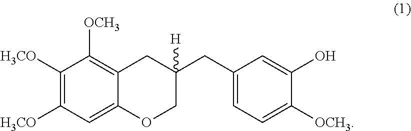 [SMILES: COc1ccc(CC2COc3cc(OC)c(OC)c(OC)c3C2)cc1O]

3. The method of claim 2, wherein the cell is a human endothelial cell.

4. The method as set forth in claim 2 comprising contacting the cell with from about 0.01 nM to about 1 μM the chromane derivative of formula (1).

5. A method of treating or preventing ocular angiogenesis-mediated disease in a subject in need thereof, the method comprising administering to the subject a chromane derivative of formula (1):

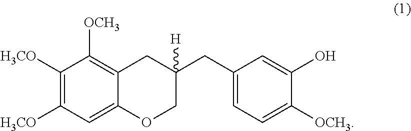 [SMILES: COc1ccc(CC2COc3cc(OC)c(OC)c(OC)c3C2)cc1O]

6. The method as set forth in claim 5 comprising administering from about 0.2 μg to about 200 μg the chromane derivative of formula (1) to the subject.

7. The method as set forth in claim 5 comprising orally administering the chromane derivative of formula (1) to the subject.

8. The method as set forth in claim 5 comprising administering the chromane derivative of formula (1) via intravitreal injection once a month to the subject.

9. The method as set forth in claim 5 comprising administering the chromane derivative of formula (1) via eye drops or eye ointment at a dosing regimen selected from the group consisting of once a day and twice a day to the subject.

10. The method as set forth in claim 5 further comprising administering an anti-vascular endothelial growth factor (anti-VEGF) agent in combination with the chromane derivative of formula (1).

11. The method as set forth in claim 10, wherein the anti-VEGF agent is selected from the group consisting of pegaptanib, ranibizumab, aflibercept, bevacizumab, brolucizumab, conbercept, Abicipar Pegol, pazopanib, regorafenib, and PAN-90806 and combinations thereof.

12. The method as set forth in claim 5, wherein the subject has a disease selected from the group consisting of retinopathy of prematurity (ROP), proliferative diabetic retinopathy (PDR), diabetic retinopathy, wet age-related macular degeneration (AMD), pathological myopia, hypertensive retinopathy, occlusive vasculitis, polypoidal choroidal vasculopathy, diabetic macular edema, uveitic macular edema, central retinal vein occlusion, branch retinal vein occlusion, corneal neovascularization, retinal neovascularization, ocular histoplasmosis, neovascular glaucoma, retinoblastoma, and combinations thereof.

13. A pharmaceutical composition comprising a chromane derivative of formula (1) for treatment or prevention of ocular angiogenesis-mediated disease:

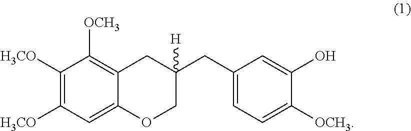 [SMILES: COc1ccc(CC2COc3cc(OC)c(OC)c(OC)c3C2)cc1O]

14. The pharmaceutical composition as set forth in claim 13, wherein the ocular angiogenesis-mediated disease is selected from the group consisting of retinopathy of prematurity (ROP), proliferative diabetic retinopathy (PDR), diabetic retinopathy, wet age-related macular degeneration (AMD), pathological myopia, hypertensive retinopathy, occlusive vasculitis, polypoidal choroidal vasculopathy, diabetic macular edema, uveitic macular edema, central retinal vein occlusion, branch retinal vein occlusion, corneal neovascularization, retinal neovascularization, ocular histoplasmosis, neovascular glaucoma, retinoblastoma, and combinations thereof.
